(19) United States
(12) Patent Application Publication
(10) Pub. No.: US 2026/0114888 A1
(43) Pub. Date: Apr. 30, 2026

(54) THROMBECTOMY SYSTEM AND METHOD OF REMOVING THROMBUS

(71) Applicant: Shifamed Holdings, LLC, Campbell, CA (US)

(72) Inventors: Aadel AL-JADDA, San Carlos, CA (US); Praveen Krishna DALA, San Ramon, CA (US); Uday ILLINDALA, Los Gatos, CA (US); Paul GUNNING, Montara, CA (US); Muralidharan SRIVATHSA, Santa Clara, CA (US); Tom SAUL, Portland, OR (US)

(21) Appl. No.: 19/123,691

(22) PCT Filed: Oct. 30, 2023
(86) PCT No.: PCT/US2023/078240

§ 371 Date: Apr. 23, 2025

Related U.S. Application Data
(60) Provisional application No. 63/381,467, filed on Oct. 28, 2022.
(60) Provisional application No. 63/381,904, filed on Nov. 1, 2022.
(60) Provisional application No. 63/477,626, filed on Dec. 29, 2022.

(51) Int. Cl. A61B 17/221; A61B 17/00; A61B 17/22; A61B 17/3203

(52) U.S. Cl. CPC A61B 17/221; A61B 17/32037; A61B 2017/00544; A61B 2017/00561; A61B 2017/00862; A61B 2017/22079; A61B 2017/2212; A61B 2217/005

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus. The system can include a rib structure for manipulating and/or urging clot material into an aspiration lumen of the catheter. The rib structure may be controlled by a clinician through a handle outside the body.

### PRIORITY CLAIM

This patent application claims priority to U.S. provisional patent application No. 63/381,467, titled “THROMBECTOMY SYSTEMS AND METHODS,” filed on Oct. 28, 2022, and U.S. provisional patent application No. 63/381,904, titled “THROMBECTOMY SYSTEM AND METHOD OF REMOVING THROMBUS,” filed on Nov. 1, 2022, and U.S. provisional patent application No. 63/477,626, titled “THROMBECTOMY SYSTEMS AND METHODS”, filed Dec. 29, 2022, all of which are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to thrombectomy systems for removing thrombus, and in various aspects to systems including aspiration and associated methods for removing a thrombus from a mammalian blood vessel.

### BACKGROUND

Thrombotic material may lead to a blockage in fluid flow within the vasculature of a mammal, such as a human. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Embolic events have historically been treated with slow-acting pharmacologic agents such as lytic therapy or catheter-directed therapy. Clinicians have turned to mechanical thrombectomy increasingly in recent years with the advent of more effective therapies. Examples of mechanical thrombectomy systems include aspiration catheters designed to enable easier navigation to the clot for removal and produce larger aspiration forces.

Existing mechanical thrombectomy systems suffer from several drawbacks, however. Systems relying on aspiration alone require larger catheters for larger clot. Softer clot may potentially be removed through a smaller profile catheter, but the morphology of clots varies and cannot be determined in advance. Indeed, clinicians oftentimes can only assess the clot after it has been removed from the body. Likewise, larger catheters cannot access smaller vessels. For this reason, clinicians must make a tradeoff between aspiration on the one hand and navigation and access on the other hand.

Even larger catheters struggle to remove organized clot because it cannot be sucked down into the relatively smaller aspiration lumen. In many cases the clinician captures the clot in the distal end of the catheter with aspiration (sometimes called “lollipopping”) and removes the catheter with the thrombus. This requires multiple introductions of the catheter in the patient which introduces significant risk of complications and adds precious time to the emergent procedure.

Other tools have been developed to remove difficult clot like clot retrievers, but these tools are not being widely adopted because of their limited effectiveness and additional costs versus aspiration or the standard of case. Some clot retrievers, which are typically formed as a wireform/mesh basket scaffolds, tend to get filled/clogged prior to target removal of thrombus burden. This can necessitate cleaning of the scraper intraoperatively, which conventionally involves withdrawing the device from the body, mechanical removal of the thrombus from the device, and re-introduction of the device to continue treatment. This process may need to be repeated several times before the target thrombus burden is removed. Such conventional approaches are painstaking and time consuming, and the thrombus that clogs the device may require large forces/impacts (e.g., striking device against a hard surface) to successfully clear the device for re-introduction. Such forces have a potential to damage the device, potentially putting the patient at risk for injury as the procedure is continued.

For the above and other reasons, many patients presenting with deep vein thrombus (DVT) are left untreated as long as the risk of limb ischemia is low. There remains the need for a device to address these and other problems with existing thrombectomy systems including, but not limited to, a fast, easy-to-use, and effective device for removing a variety of clot morphologies. There remains the need for a device for removing a variety of clot morphologies in smaller vessels and deeper in the peripheral vasculature. There remains the need for a device for removing a variety of clot morphologies in a single pass.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1 and 1A illustrate various views of a distal portion of a thrombus removal system including a funnel in accordance with various embodiments of the present technology.

FIGS. 2A-2E illustrate plan views of various configurations of irrigation ports and fluid streams for use with the catheter of FIG. 1A.

FIGS. 3A-3H illustrate an elevation view of various configurations of irrigation ports and fluid streams for use with the catheter of FIG. 1A.

FIGS. 4A-4C illustrate various embodiments of a thrombus removal system including a saline source, an aspiration system, and one or more controls for controlling irrigation and/or aspiration of the system.

FIGS. 4D-4E illustrate an introducer system for introducing a medical device into the vasculature of a subject.

FIGS. 5A-5C illustrate a thrombectomy catheter including a scaffold according to embodiments of the present disclosure.

FIGS. 6A-6D illustrate a thrombectomy catheter including a scaffold according to embodiments of the present disclosure.

FIG. 7 is a flowchart showing a method of performing a thrombectomy procedure.

FIGS. 8A-8D illustrate one embodiment of a thrombectomy device.

FIG. 9 illustrates a thrombectomy device according to embodiments of the present disclosure.

FIG. 10 illustrates a thrombectomy device according to embodiments of the present disclosure.

FIG. 11 illustrates a thrombectomy device according to embodiments of the present disclosure.

FIG. 12 illustrates a thrombectomy device according to embodiments of the present disclosure.

FIG. 13 illustrates a thrombectomy device according to embodiments of the present disclosure.

### SUMMARY OF THE DISCLOSURE

A thrombus removal system is provided, comprising mechanisms to capture and remove clot from the body. In various embodiments, the system includes mechanically actuated arms for pulling clot into the aspiration lumen. In various embodiments, the mechanically actuated arms are formed within an expandable distal end of the catheter. In various embodiments, the expandable distal end of the catheter is formed as a funnel. In various embodiments, the catheter includes ports for delivering fluid jets to disrupt or break up the receiving clot such that the clot material can be aspirated through a smaller lumen. In various embodiments, a thrombus removal system is disclosed that (further) includes a scaffold or wireform structure that is adapted to be pulled along a vessel and remove clot thereby. In various embodiments, the thrombus removal system provides 1). thrombus removal and 2) in situ simultaneous and/or sequential cleaning of the scaffold. For example, irrigation (jets) can be used as a means of (pressure) washing the scraper scaffold. The vessel can be protected by presenting the scraper scaffold to the irrigation within an interior portion of the thrombectomy catheter.

In one aspect, a thrombus removal device is provided, comprising: an elongate shaft; at least one aspiration lumen in the elongate shaft; a funnel disposed at or near a distal end of the elongate shaft; and an expandable scaffold assembly deployable distally from the funnel, the scaffold assembly being configured to capture clots; wherein an axial position of the scaffold assembly relative to the funnel is adjustable to bring the scaffold assembly near or into contact with the funnel to aspirate clots in the scaffold assembly through the funnel.

In some aspects, the system includes one or more tethers coupled to the scaffold assembly.

In some aspects, the one or more tethers are routed internally to the elongate shaft.

In other aspects, the one or more tethers are routed internally to the funnel.

In one aspect, the one or more tethers are routed externally to the elongate shaft.

In other aspects, the one or more tethers are routed externally to the funnel.

In some aspects, the one or more tethers are configured to adjust the axial position of the scaffold assembly relative to the funnel.

In one aspect, the funnel is configured to engage with the scaffold assembly.

In other aspects, the funnel is configured to be inserted into the scaffold assembly.

In some aspects, the scaffold assembly is configured to be inserted into the funnel.

In another aspect, a proximal opening of the scaffold assembly is configured to mate with the funnel.

In some aspects, the thrombus removal device is configured to produce one or more fluid streams within the funnel to interact with clots in the funnel or scaffold assembly.

In some aspects, the scaffold assembly is configured to evert into the funnel.

In other aspects, manipulation of one or more tethers coupled to the scaffold is configured to evert the scaffold assembly into the funnel.

In some aspects, the scaffold assembly comprises a superelastic material.

In one aspect, the scaffold assembly is configured to self-expand from a delivery configuration to a deployed configuration.

A thrombectomy method is provided, comprising: positioning a thrombectomy device near a target thrombus in a patient; deploying a scaffold assembly from the thrombectomy device; capturing the target thrombus in the scaffold assembly; positioning a funnel of the thrombectomy device near or in contact with the scaffold assembly; and aspirating the target thrombus from the scaffold assembly through the funnel with the thrombectomy device.

In some aspects, the thrombectomy device is positioned proximal to the target thrombus.

In other aspects, the thrombectomy device is positioned distal to the target thrombus.

In one aspect, the scaffold assembly is deployed distal to the target thrombus.

In some aspects, capturing the target thrombus further comprises moving the thrombectomy device and the scaffold assembly proximally to capture the target thrombus.

In some aspects, capturing the target thrombus further comprises moving the scaffold assembly proximally relative to the thrombectomy device to capture the target thrombus.

In another aspect, moving the scaffold assembly proximally further comprises manipulating one or more tethers coupled to the scaffold assembly.

In some aspects, the method further comprises engaging the thrombectomy device with the scaffold assembly.

In other aspects, the method includes engaging a funnel of the thrombectomy device with the scaffold assembly.

In some aspects, engaging the thrombectomy device with the scaffold assembly further comprises pulling the scaffold assembly near, within, or into contact with the thrombectomy device with one or more tethers coupled to the scaffold assembly.

In other aspects, engaging the thrombectomy device with the scaffold assembly further comprises advancing the thrombectomy device near, within, or into contact with the scaffold assembly.

In some aspects, the method includes breaking up the target thrombus with one or more jets of the thrombectomy device.

In some aspects, the method further comprises everting the scaffold assembly into the thrombectomy device.

In another aspect, the method includes everting the scaffold assembly into a funnel of the thrombectomy device.

In some aspects, everting the scaffold assembly comprises manipulating one or more tethers coupled to the scaffold assembly.

A thrombectomy device for removing thrombus in a patient is provided, the thrombectomy device comprising: an elongate shaft extending along a longitudinal axis and having a distal end formed generally in a plane that is at an angle with respect to the longitudinal axis; an aspiration lumen disposed in the elongate shaft and coupled to an aspiration source; at least one irrigation lumen disposed in the elongate shaft and coupled to an irrigation source; and a plurality of apertures disposed near the distal end and fluidly coupled to the at least one irrigation lumen, wherein the plurality of apertures are configured to produce a plurality of fluid streams in a plane that is generally parallel to the angle of the distal end.

In some aspects, at least two apertures sized and shaped to direct respective fluid streams in the plane.

In another aspect, the device has at least two apertures sized and shaped to direct respective fluid streams along intersecting paths toward an intersection region.

In some aspects, the fluid streams are directed along intersecting paths toward at least two intersection regions.

In other aspects, the intersecting region is within the plane.

In some aspects, the intersecting region is distal to the plane.

In one aspect, the intersecting region is proximal to the plane.

In other aspects, the device includes a guidewire lumen extending proximally from a distal region of catheter along at least a portion of the elongate shaft.

In another aspect, the elongate shaft has an outer wall encompassing the aspiration lumen, the irrigation lumen, and the guidewire lumen, wherein the aspiration lumen is formed by an aspiration wall, the guidewire lumen is formed by a guidewire wall, and the irrigation lumen is formed from the portions of the elongate shaft that are internal to the outer wall and external to the aspiration wall and the guidewire wall.

In some aspects, the irrigation lumen comprises a partition extending along the elongate shaft, the partition providing an isolated irrigation lumen portion fluidly coupled to one irrigation aperture.

In one aspect, the irrigation lumen further comprises a second irrigation lumen portion fluidly coupled to at least two irrigation apertures.

In one aspect, the device includes a pressure sensor.

In one aspect, the pressure sensor is a fluid column pressure sensor or a MEMs pressure sensor.

In another aspect, the apertures are configured to produce more than one fluid stream spray pattern.

In some aspects, the fluid stream spray pattern is selected from the group consisting of solid stream, solid cone, hollow cone, flat fan, and a mist.

In another aspect, one or more of the apertures have a first size and one or more of the apertures have a second size.

In some aspects, a distal edge of the device is beveled.

In another aspect, a distal edge of the device is convex.

In some aspects, a distal edge of the device is concave.

In some aspect, the distal end of the elongate shaft is formed from a distal wall, wherein the elongate shaft further comprises an outer wall encompassing the aspiration lumen and the irrigation lumen, wherein the aspiration lumen is formed by an aspiration wall extending along the elongate shaft, and the irrigation lumen is formed from the portions of the elongate shaft that are internal to the outer wall and external to the aspiration wall and the distal wall.

### DETAILED DESCRIPTION

This application is related to disclosure in International Application No. PCT/US2021/020915, filed Mar. 4, 2021 (the '915 application), International Application No. PCT/US2022/033024, filed Jun. 10, 2022 (the '024 application), provisional application no. 63/381,467, filed Oct. 28, 2022, provisional No. 63/381,019, filed Oct. 26, 2022, and provisional No. 63/373,413, filed Aug. 24, 2022, the entire disclosures of which are incorporated by reference herein for all purposes. These applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

Any headings provided herein are for convenience only and do not interpret the scope or meaning of the claimed present technology.

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments through a small lumen of the catheter out of the patient's body. In examples including pressurized fluid streams (e.g., jets), the fluid streams may disrupt, cut, or macerate thrombus. Fragmentation helps to prevent clogging of the aspiration lumen and/or allows the thrombus removal system to macerate large, firm clots that otherwise could not be aspirated. As used herein, “thrombus”, “embolism”, and “clot” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

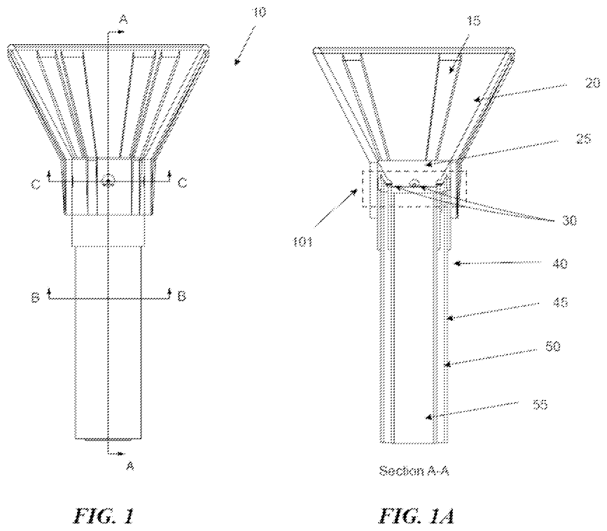

FIG. 1 illustrates a distal portion 10 of an exemplary thrombus removal system according to an embodiment of the present technology. FIG. 1A Section A-A illustrates an elevation sectional view of the distal portion. The example section A-A in FIG. 1A depicts an exemplary funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

The dimensions and configurations of the system may depend on the application. Conventional thrombectomy systems are sized based on the target location. For example, a larger bore aspiration catheter can remove a higher clot burden but may be too large to access anything other than main vessels. Conversely, smaller aspiration catheters may be required for small vessels, such as the small vessels past the pulmonary trunk or peripheral vasculature and deep veins. The system described herein allows for different configurations to avoid common tradeoffs in conventional systems.

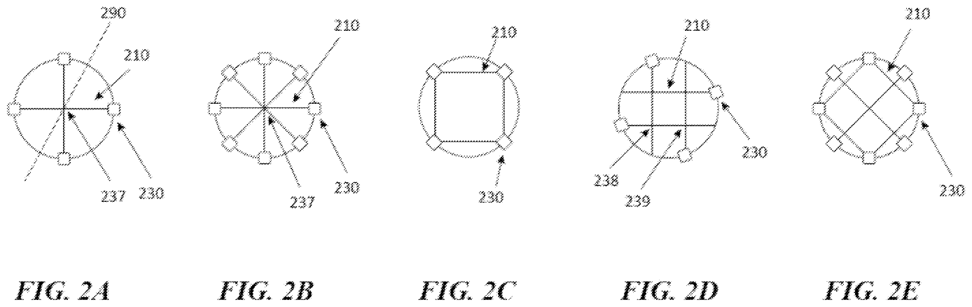

Turning to FIGS. 2A to 2E, in some embodiments, port(s) 230 is formed near the opening of the aspiration lumen to direct the fluid flow along a selected path. In the exemplary embodiment, the catheter includes a funnel and the ports are formed at the base of the funnel where clot is pulled into the aspiration lumen.

FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

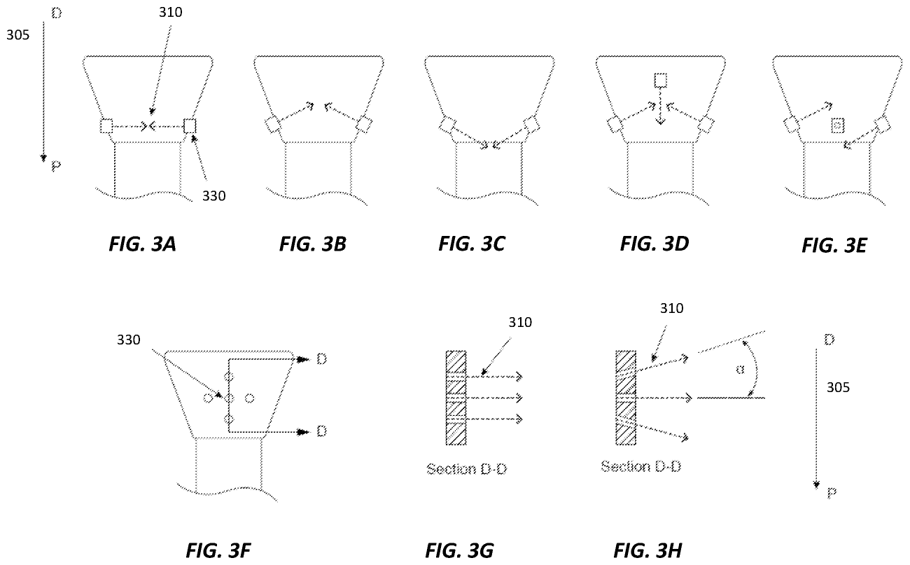

FIGS. 3A-3H depict various configurations of fluid streams 410 that are directed from respective ports 430. A fluid stream 410 can be directed along a path that is substantially orthogonal, proximal, and/or distal to the flow axis 405. In some embodiments, at least two fluid streams are directed in different directions with respect to the flow axis 305. In some embodiments, at least two fluid streams are directed in a same direction (e.g., proximally) with respect to the flow axis 305. In some embodiments, at least a first fluid stream is directed orthogonally, at least a second fluid stream is directed proximally, and at least a third fluid stream is directed distally with respect to the flow axis 305. An angle a may characterize an angle that a fluid stream 310 is directed with respect to an axis that is orthogonal to the flow axis 305 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 330 in a nominal direction (e.g., distally) is deflected along an altered path (e.g., proximally) by (e.g., suction) pressure generated by the aspiration mechanism during operation.

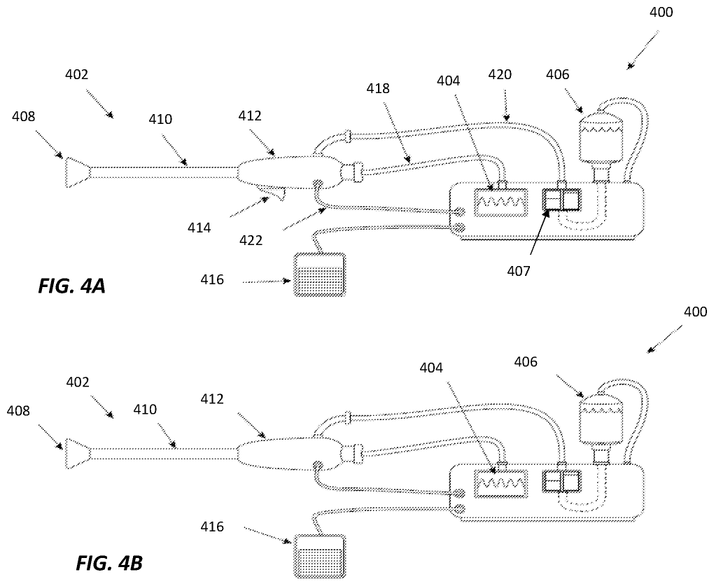

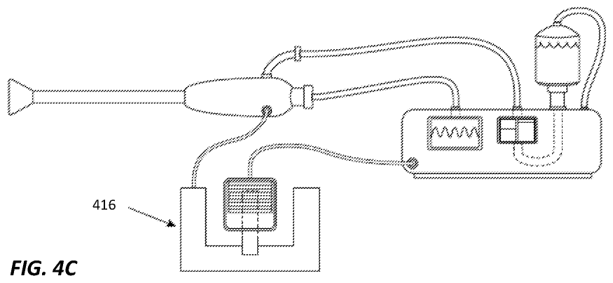

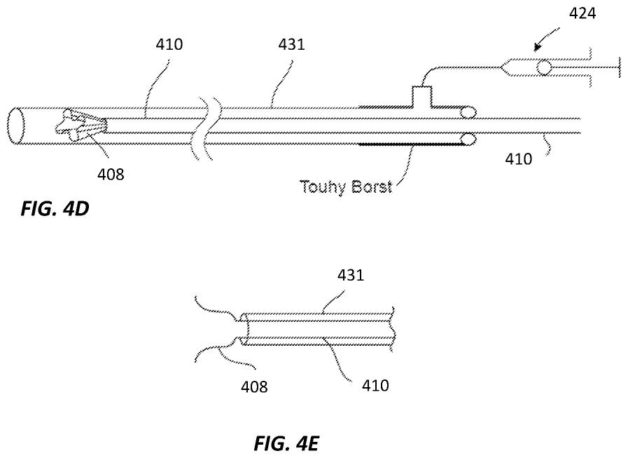

FIGS. 4A-4C illustrate various configurations of a thrombus removal system 400, including a thrombus removal device, 402, a vacuum source and cannister 404, a fluid source 406, and a pump 407. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 402 can include a funnel 408, a flexible shaft 410, a handle 412, and one or more controls 414 and 416. For example, in the embodiment shown in FIG. 4A, the device can include a finger switch or trigger 414 and a foot pedal or switch 416. These can be used to control aspiration and irrigation, respectively. Alternatively, as shown in the embodiment of FIG. 4B, the device can include only a foot switch 416, which can be used to control both functions, or in FIG. 4C, the device can include only an overpedal 416, also used to control both functions. It is also contemplated that an embodiment could include only a finger switch to control both aspiration and irrigation functions. As shown in FIG. 4A, the vacuum source and cannister 404 can be coupled to the aspiration lumen of the device with a vacuum line 418. Any clots or other debris removed from a patient during therapy can be received by, and stored in, the vacuum cannister 404 for later disposal. Similarly, the fluid source 406 (e.g., a saline bag) can be coupled to the fluid lumens of the device with a fluid line 420 for delivery of high-pressure and velocity fluid streams or jets at the base of funnel 408 to fragment thrombus material engaged by funnel 408, as described above.

Still referring to FIG. 4A, electronics line 422 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

In FIG. 4D, a system assembly is shown including a funnel 408 and a flexible shaft 410 of a thrombus removal device inserted into a steerable introducer catheter 431. A hub assembly such as a Touhy Borst is shown which can provide access for a medical device into the steerable introducer catheter and include an injection port for fluidic connection to the contrast injector 424. In this embodiment, injection of contrast from the injector 424 into the hub assembly provides the contrast agent into the annular space between the introducer catheter 431 and the thrombus removal device (e.g., the shaft of the thrombus removal device).

FIG. 4E shows the funnel 408 of the thrombus removal device axially disposed out of a distal end of the introducer catheter 431. In this example, contrast delivered by the injector 424 into the annular space can still be delivered into the patient, even when the funnel is in a deployed configuration. In some examples, the funnel can disperse the contrast agent as it's delivered past the funnel from the annular space.

### Arterial Applications

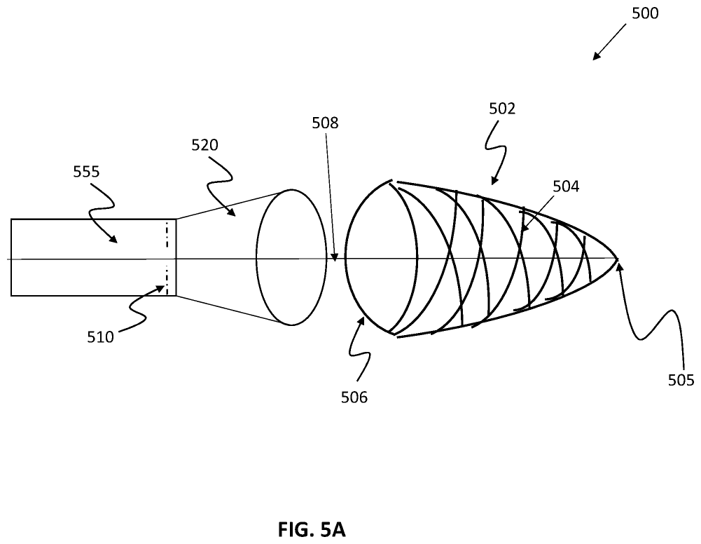

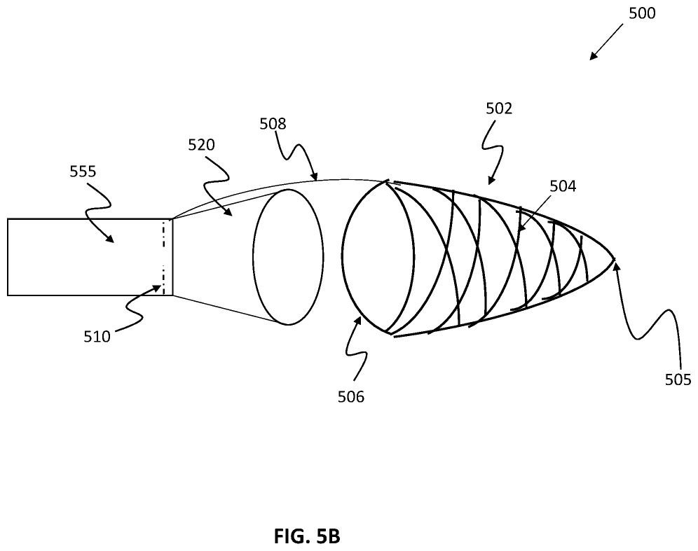

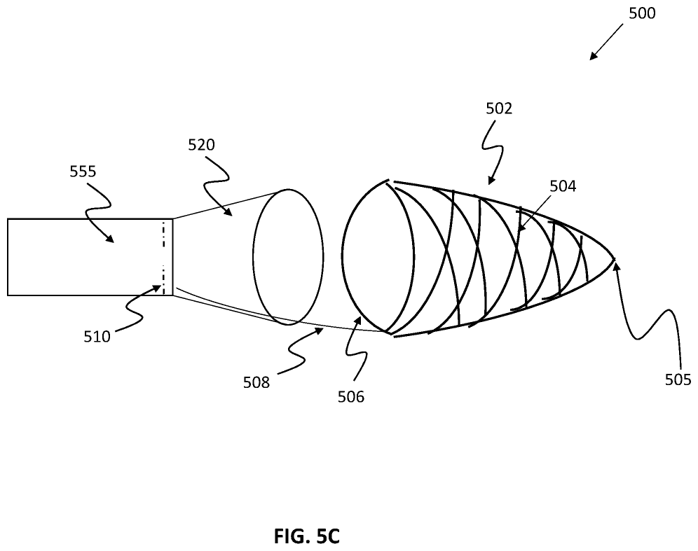

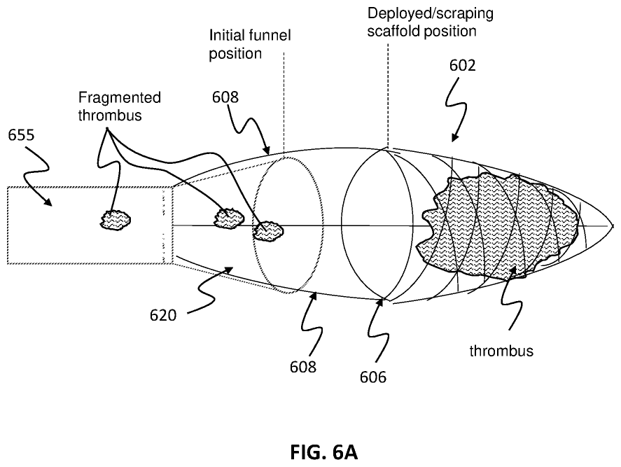

In FIGS. 5A-5C, several approaches can be employed for interfacing a thrombectomy catheter 500 with a scaffold 502 (also referred to herein as a “windsock”). In general, in operation the scaffold positioned distal to the thrombectomy catheter. In some examples, the scaffold 502 has a deployed configuration that is distal to a funnel 520 of the thrombectomy catheter device. As described above, any of the thrombectomy catheter embodiments that include a scaffold can include an aspiration lumen 555 coupled to an aspiration source for pulling clots into the funnel 520, and one or more fluid streams or jets 510 configured to break up or macerated the clot for removal from the patient.

The scaffold can comprise a frame 504 which can provide structural support for the scaffold and allow the scaffold to assume its deployed configuration. The frame can provide a desired shape for the scaffold, such as a conical shape, cylindrical shape, spherical shape, oval shape, or any other desired shape. The frame can further define a proximal opening 506 which can be expanded (self-expanded) to allow thrombus material to enter the scaffold. In some aspects, only the proximal opening 506 of the scaffold is configured to allow thrombus material to enter into the scaffold. The proximal opening can also be configured to expand to assume a desired shape. In some embodiments, the proximal opening is configured to assume a circular cross-sectional shape that is well suited and configured to conform to or reside against the interior lumens of the vasculature, such as arteries and veins. The distal end of the scaffold can be closed or sealed to retain captured thrombus.

In some embodiments, the frame is compressible and expandable which allows the scaffold to assume a compressed delivery configuration (e.g., within the thrombectomy catheter, within a stylet catheter, or within another introducer sheath) and which also allows the scaffold to assume its deployed/expanded configuration as shown. In some embodiments, the frame can comprise a shape memory or superelastic material such as Nitinol. The superelastic material frame can allow the frame to self-expand from the delivery configuration to the deployed configuration. The frame can be laser-cut from a nitinol sheet or tube. Alternatively, the frame can comprise a mesh or even a plurality of wires, cords, or cables. In some aspects, the frame can support or hold a separate mesh or filter. In some aspects, the frame and/or mesh is configured to allow blood to pass but not allow clots or thrombus material to pass. In some aspects, the frame and/or mesh can have a pore or opening size on the order of 40 microns, or alternatively, between 50 to 300 microns, to allow blood but not clots or thrombus material to pass.

The scaffold can be tethered to the thrombectomy catheter by one or more wires or tethers 508. The tethers 508 can be routed along the catheter along an exterior or interior aspect (e.g., in lumens, such as the lumens of the thrombectomy catheter shown in FIGS. 1 and 1A) of the thrombectomy catheter. For example, in the embodiment of FIG. 5A, the scaffold 502 can be attached to the thrombectomy catheter with a tether 508 centrally placed within the scaffold and coupled to a distal portion 505 of the scaffold. In some aspects, the central tether of FIG. 5A can be routed through a central channel of the thrombectomy catheter such as the aspiration lumen 555 of the thrombectomy catheter. In other embodiments, not shown, the central tether can be routed along an exterior of the thrombectomy catheter, or within other lumens of the thrombectomy catheter.

In the example of FIG. 5B, the tether 508 can be routed to an exterior of the thrombectomy catheter, such as exterior to the funnel 520 and the catheter shaft. In some aspects, the tether can be coupled to the proximal opening 506 of the frame 504. Alternatively, the tether can be coupled to the distal end 505 of the scaffold.

FIG. 5C shows the tether 508 routed inside the funnel into a lumen of the thrombectomy catheter device. For example the tether can run proximally into the aspiration lumen of the device, or into a separate lumen in the catheter shaft, such as a fluid lumen or an auxiliary lumen dedicate for routing the scaffold tether(s).

In any of the embodiments disclosed herein, any combination of tethers can be used to couple to the scaffold, including central tethers, exterior tethers, internally routed tethers, tethers that attach to the proximal opening or distal end of the scaffold, or even attachment points to the scaffold frame itself.

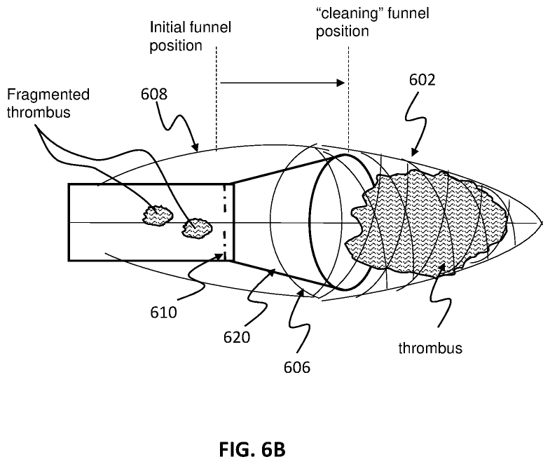

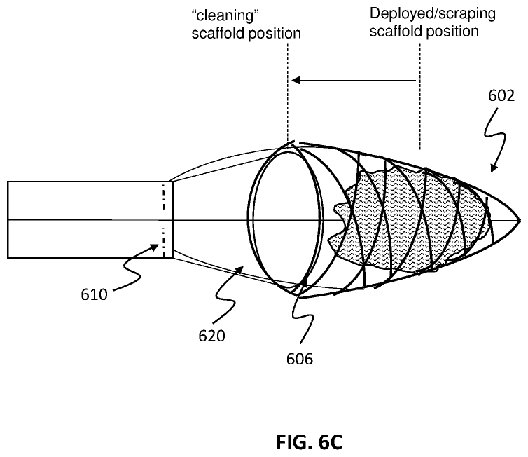

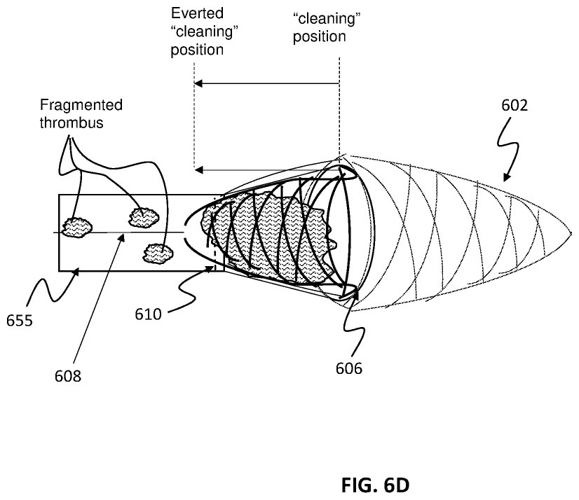

The tethers enable manipulation of a position of the scaffold with respect to the thrombectomy catheter, and/or a configuration (e.g., shape) of the scaffold. In some examples, the tethers can be manipulated or controlled to place the scaffold in a deployed or scraping configuration relative to the initial position of the thrombectomy catheter funnel. As shown in FIG. 6A, the one or more tethers can be advanced or released to deploy the scaffold 602 distally to the funnel 620 of the thrombectomy catheter. In the illustrated example, a thrombus (or multiple thrombi) is captured inside the scaffold 602. In some embodiments, the scaffold is deployed distally to or beyond a target thrombus, and the entire assembly (e.g., the thrombectomy catheter and the scaffold) can be pulled proximally to capture the thrombus in the scaffold through the proximal opening 606. This example maintains the relative distance between the scaffold and the funnel.

Alternatively, referring to FIG. 6B-6C, the relative position between the scaffold and the funnel can be adjusted, either by advancing the thrombectomy catheter and funnel towards the scaffold and captured thrombus (FIG. 6B), or by manipulating the tethers (e.g., pulling the tethers) to pull the scaffold towards the funnel 620 of the thrombectomy catheter (FIG. 6C). In some embodiments, the relative axial adjustment between the scaffold and the thrombectomy catheter results in the funnel 620 entering into the scaffold through the proximal opening 606. In other embodiments, the funnel 620 “docks” or mates with the proximal opening.

FIG. 6B shows the relative position of the funnel moving from the initial funnel position to the “cleaning” funnel position in which the funnel is engaged with or inside the scaffold. In some embodiments, a funnel of the thrombectomy catheter is sized and shaped to fit within the scaffold and “scrape” along an interior aspect thereof, to dislodge clot that may be adhered thereto.

In FIG. 6C, the position of the thrombectomy catheter/funnel is not moved, but instead the tethers are manipulated to pull the scaffold from the deployed/scraping scaffold position to a “cleaning” or docked scaffold position that engages with the funnel 620.

In any of the above embodiments, the aspiration and jetting (e.g., jets or fluid streams 610) of the thrombectomy catheter can be active to pull thrombus from the scaffold into the funnel and into the jet plane of fluid streams 610 to macerate or break up the thrombus. In some aspects, jetting and/or aspiration is only active when the scaffold is docked or mated with the funnel in the aforementioned “cleaning” position. In various embodiments, the scaffold may interface with a distal end of the thrombectomy catheter to present captured clot for aspiration and/or irrigation by the thrombectomy catheter. In FIG. 6D, the scaffold may be withdrawn further (e.g., everted) to an interior of the thrombectomy catheter funnel, enabling the interior surfaces thereof or pulled proximally and presented to the irrigation jets 610 and/or aspiration lumen 655 of the thrombectomy catheter. This movement may be accomplished by manipulation of tether(s) positioned at select regions of the scaffold, for example the proximal mouth and the distal end. In some embodiments, the scaffold includes “docking” a relatively stiff (proximal) mouth of the scaffold at the distal end of the thrombectomy catheter (funnel).

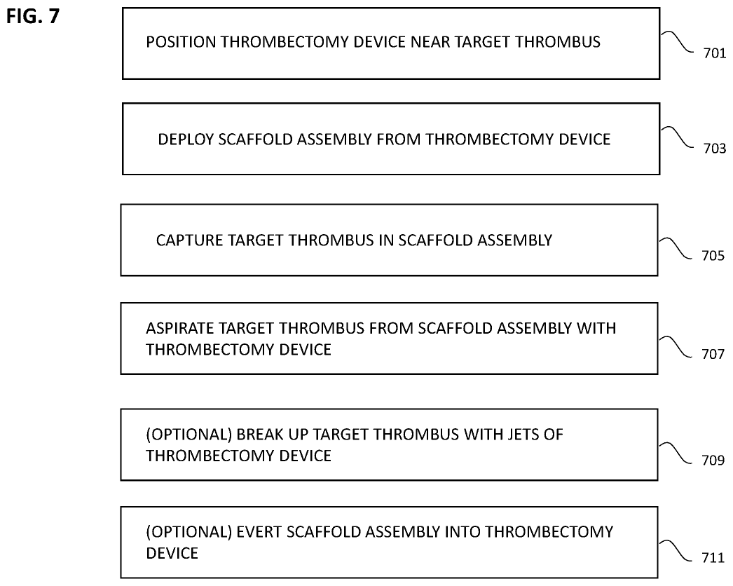

FIG. 7 is a flowchart describing a thrombectomy method according to the embodiments described above in FIGS. 5A-6D. At step 701 of the flowchart, the method can include positioning a thrombectomy device near a target thrombus. The thrombectomy device can be advanced along a guidewire, or alternatively, introduced with an introducer catheter or sheath. The thrombus may be located in an artery or vein of a subject, for example. In some embodiments, the thrombectomy device may be positioned proximal to the target thrombus. In other embodiments, the thrombectomy device may be positioned distal to the target thrombus (e.g., past the target thrombus). In some aspects, the guidewire is inserted through the target thrombus and the thrombectomy catheter is carried by the guidewire across the target thrombus.

At step 703, the method can include deploying a scaffold assembly from the thrombectomy device. In some embodiments, the scaffold assembly is deployed from a compressed or collapsed delivery configuration to an expanded or deployed configuration. The scaffold assembly may be self-expanding, such as with a shape memory effect. In some embodiments, the scaffold assembly is deployed from within the thrombectomy device itself, such as within a stylet catheter that is carried by the thrombectomy device. In other embodiments, both the scaffold assembly and the thrombectomy device are deployed from a separate introducer catheter or sheath. The scaffold assembly is typically deployed distal to the target thrombus. In one embodiment, the thrombectomy device is positioned proximal to the thrombus, and the scaffold assembly is deployed distal to the target thrombus. In another embodiment, the thrombectomy device is positioned distal to the thrombus, and the scaffold assembly is deployed distal to the target thrombus. The thrombectomy device may be retracted proximally in this embodiment to place the thrombectomy catheter proximal to the thrombus and the scaffold assembly distal to the target thrombus.

At step 705, the method can include capturing the target thrombus in the scaffold assembly. In some examples, the entire assembly, including the thrombectomy device and the scaffold assembly can be pulled or moved proximally to capture the target thrombus in the scaffold assembly, such as through a proximal opening of the scaffold assembly. In other embodiments, tethers attached to the scaffold assembly can be manipulated to adjust a position of the scaffold assembly relative to the thrombectomy device (e.g., pulling the scaffold assembly towards the thrombectomy device).

At step 707, the method can include aspirating the target thrombus from the scaffold assembly with the thrombectomy device. For example, the clot or thrombus can be aspirated into an aspiration lumen of the thrombectomy device. In some examples, aspirating the clot from the scaffold assembly requires engaging, mating, or docking the scaffold assembly with the thrombectomy device. This can include engaging, mating, or docking the scaffold assembly with a funnel of the thrombectomy device. However, it should be understood that the scaffold assembly can be engaged, mated, or docked directly with an aspiration lumen of the thrombectomy device without requiring a funnel on the device. Additionally, direct contact between the thrombectomy device and the scaffold assembly is not required, as aspiration from the thrombectomy catheter may pull the thrombus from the scaffold into the device if the scaffold and/or thrombus are positioned close enough to the aspiration lumen of the device. As described above, engaging, mating, or docking the scaffold assembly with the thrombectomy device can include adjusting a relative position between the scaffold assembly and the thrombectomy catheter, such as by pulling the scaffold assembly near, within, or into contact with the thrombectomy device (e.g., with tethers), or alternatively pushing or advancing the thrombectomy device near, within, or into contact with the scaffold assembly.

At optional step 709, the method can include breaking up the target thrombus with jets of the thrombectomy device. As described herein, the thrombectomy device may be configured to direct one or more fluid jets or fluid streams towards a thrombus, such as within an aspiration lumen or a funnel of the device. The jets or fluid streams may be intersecting.

At optional step 711, the method can also include everting the scaffold assembly into the thrombectomy device, such as into the funnel of the thrombectomy device. In some examples, everting the scaffold assembly can comprise manipulating one or more tethers coupled to the scaffold assembly to evert the scaffold assembly into the funnel.

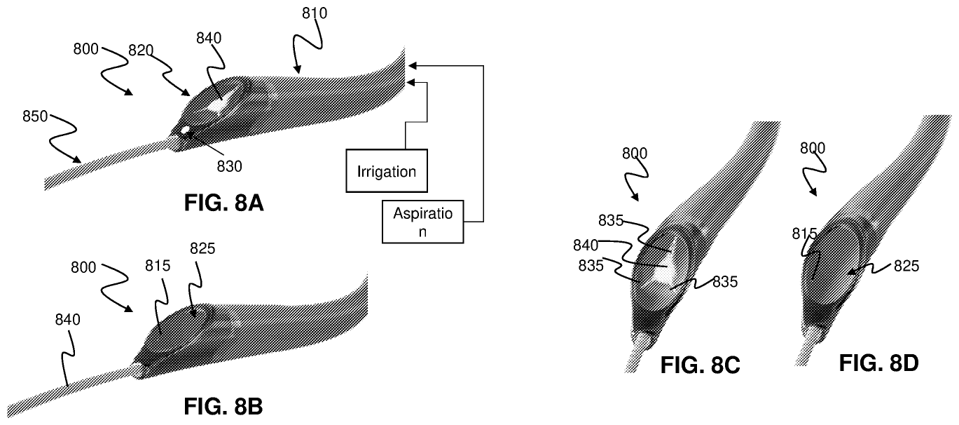

FIG. 8A illustrates a thrombectomy device 800 according to embodiments of the present disclosure. As shown in FIG. 8A, the thrombectomy device 800 includes a proximal portion 810, a distal beveled portion 820, and jets, orifices, or nozzles configured to produce colliding irrigation streams 840. The thrombectomy device 800 accommodates a guidewire 850. The thrombectomy device 800 is operatively coupled to an irrigation source and an aspiration source to provide irrigation fluid and vacuum pressure, respectively. In some implementations, the thrombectomy devices described herein can have a diameter up to and including 7 Fr outer diameter (OD) and 4 Fr inner diameter (ID).

In any embodiment herein, the thrombectomy device can be configured to deliver a contrast agent into tissue. For example, the thrombectomy device can be configured to deliver contrast agent through an aspiration lumen of the device, through irrigation lumens, through auxiliary lumens, or in an annular space between the thrombectomy device and an introducer sheath/catheter.

In some embodiments, the thrombectomy device 800 can include a pressure sensor 830. The pressure sensor can be positioned near a distal beveled portion 820 of the device. The pressure sensor can comprise a fluid column, a MEMs pressure sensor, or any other pressure sensor as known in the art. The pressure sensor can be configured or used to track progress of a procedure based on changes in pressure. The changes in pressure can be, for example, measured according to the ankle brachial index or other clinically performed measures. The ankle-brachial index test compares the blood pressure measured at the ankle with the blood pressure measured at the arm.

FIG. 8B illustrates a thrombectomy device 800 according to embodiments of the present disclosure. As shown in FIG. 8B, the thrombectomy device 800 includes an aspiration lumen 825 fluidly coupled to the aspiration source, and one or more irrigation apertures 815 fluidly coupled to the irrigation source and adapted to direct irrigation streams. The aspiration lumen of the FIG. 8B embodiment can be included in any of the thrombectomy catheters described herein. The thrombectomy device 800 accommodates a guidewire 840.

FIG. 8C illustrates a thrombectomy device 800 according to embodiments of the present disclosure. As shown in FIG. 8C, the thrombectomy device 800 comprises a plurality of irrigation streams 835 that intersect in an intersection region 840.

FIG. 8D illustrates a thrombectomy device 800 according to embodiments of the present disclosure. As shown in FIG. 8D, the thrombectomy device 800 includes an aspiration lumen 825 fluidly coupled to the aspiration source, and one or more irrigation apertures 815 fluidly coupled to the irrigation source and adapted to direct irrigation streams.

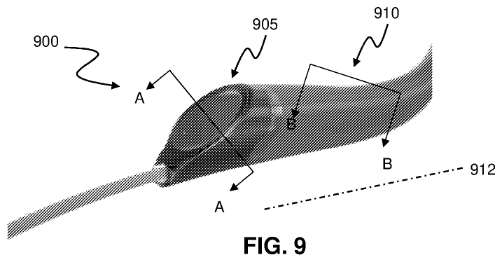

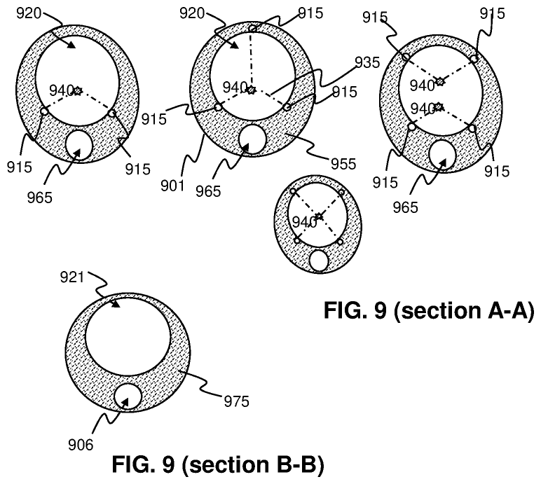

FIG. 9 illustrates a thrombectomy device 900 according to embodiments of the present disclosure. As shown in FIG. 9, thrombectomy device 900 comprises a distal portion 905 (e.g., tip) and a more proximal portion 910. The distal portion 905 may include a distal end that is angled (e.g., beveled, or skived) with respect to the longitudinal axis 912 of the thrombectomy device 900. An angled end may improve the capability of the thrombectomy device 900 to advance through a patient vasculature along the guidewire without undesirable interaction with patient tissue. The angled end may advantageously increase a cross-sectional area (e.g., elliptical) of the aspiration lumen that is presented to an adjacent clot, compared with the cross-sectional area (e.g., circular) of the aspiration lumen in more proximal portions of the thrombectomy device 900. Various configurations of irrigation apertures 915 directing fluid streams 935 toward intersections regions 940 are depicted in FIG. 9 section view A-A of the distal portion 905.

As shown in the section view A-A, the irrigation apertures 915 can be arranged about the inner perimeter of aspiration lumen 920. Thrombectomy device 900 can have 2, 3, 4, or more irrigation apertures 915. The irrigation apertures 915 can direct fluid streams to intersect at 1 or more intersection regions 940. The intersection region(s) can be positioned at selected regions of the aspiration lumen—for example, biased toward the guidewire lumen, centrally located, and/or biased away from the guidewire lumen. The irrigation apertures 915 are fluidly coupled to an irrigation lumen 955 (shown in pattern) that is defined by the internal (e.g., interstitial) spaces that are within the outer wall 901 of the thrombectomy catheter and external to the outer diameters of the aspiration lumen 920 and the guidewire lumen 965. Section view A-A is taken in a plane that is generally parallel to the (angled) distal end of the thrombectomy device 900.

As shown in section A-A, irrigation streams 935 are directed along paths that are generally within the plane that is parallel to the distal end of the thrombectomy device 900. In some embodiments the irrigation apertures 915 are at the distal end of the thrombectomy device 900. In some embodiments the irrigation apertures 915 are set back from the end by some distance—for example set back at a distance that is within a range from about 1 mm to about 30 mm from the distal end. The irrigation aperture can be formed to generate a fluid stream that is a (e.g., columnar) jet, generally conical, or generally fanned (e.g., planar).

A more proximal portion 910 of the thrombectomy device 900 is shown in section B-B, which is taken generally orthogonal to the longitudinal axis 912. The section view B-B depicts an aspiration lumen 921, a guidewire lumen 906, and irrigation lumen 975. In some embodiments these lumens can maintain a substantially similar arrangement that extends proximally to a proximal end of the thrombectomy device 900, that is fluidly coupled to the irrigation source and the aspiration source. In some embodiments the guidewire lumen 906 extends along the length of the thrombectomy device 900. In some embodiments the guidewire lumen 906 extends along a portion of the thrombectomy device 900 (e.g., in a rapid exchange configuration). The guidewire lumen can be sized to accommodate guidewires in a range of sizes —for example, 0.014 inches or 0.018 inches.

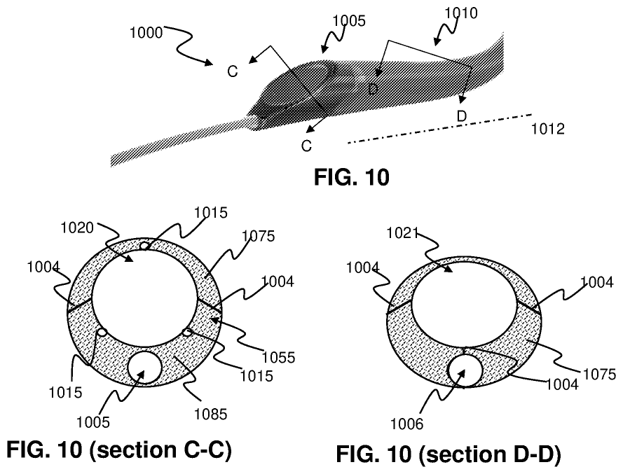

The irrigation lumen of thrombectomy devices of the present disclosure can include one or more partitions. The partitions can be placed direct flow in a selected (pre-defined) manner from the irrigation source to a given irrigation aperture(s). FIG. 10 illustrates a thrombectomy device 1000 according to embodiments of the present disclosure. As shown in FIG. 10, thrombectomy device 1000 comprises a distal portion 1005 and a proximal portion 1010 along longitudinal axis 1012. A section view C-C of the distal portion depicts aspiration lumen 1020, guidewire lumen 1005, irrigation apertures 1015 and an irrigation lumen 1055 fluidly coupled thereto that is divided into (e.g., two) portions by partitions 1004. As shown in the section view C-C, the topmost irrigation aperture 1015 is fed by approximately the top half portion 1075 of the irrigation lumen 1055, while the lower two irrigation apertures 1015 share the divided bottom portion 1085. Other arrangements of partitions are possible—for example, rather than at least two irrigation apertures 1015 sharing an irrigation lumen portion, each irrigation aperture(s) 1015 can be fed by dedicated irrigation lumen portion(s). In some embodiments the thrombectomy device 1000 includes a control (not shown) that is programmed to selectively address (actuate) individual irrigation lumen portions. The addressing of individual irrigation lumen portions can be made according to distinct frequencies, intervals, and irrigation source pressures (e.g., per irrigation lumen portion). The irrigation lumen portions and/or irrigation apertures can be sized and shaped to provide different operational (i) (e.g., average) flow velocity, (ii) (e.g., average) fluid pressure for the fluid streams generated by the irrigation apertures.

As shown in section view D-D of FIG. 10, a proximal portion 1010 of the thrombectomy device 1000 includes an aspiration lumen 1021, a guidewire lumen 1006, and an irrigation lumen 1075 that is partitioned (by 1004) into several irrigation lumen portions. In some embodiments, a same number of irrigation lumen portions are formed in the proximal section 1010 and the distal section 1005. In some embodiments, a different number of irrigation lumen portions are formed in the proximal section 1010 (e.g., 3 portions) and the distal section 1005 (e.g., 2 portions).

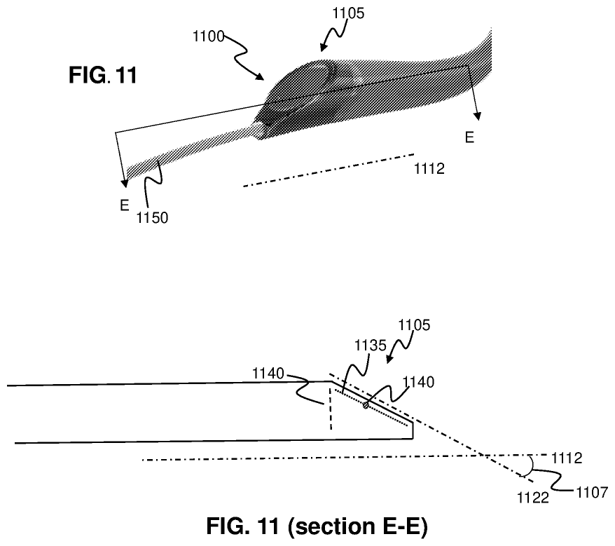

FIG. 11 illustrates a thrombectomy device 1100 according to embodiments of the present disclosure. As shown in FIG. 11, thrombectomy device 1100 comprises an elongate shaft that extends along a longitudinal axis 1112 and can accommodate a guidewire 1150. Section view E-E illustrates a face of distal portion 1105 formed generally in a plane 1122 that is angled (1107) with respect to the longitudinal axis 1112. As depicted in section view E-E irrigation streams 1135 can be directed from irrigation apertures toward one or more intersection regions 740 that are within a plane that is generally parallel to 1122. Additionally or alternatively, irrigation streams can be directed off-axis with respect to the plane 1122—for example orthogonally toward the longitudinal axis 1112. Other directions of fluid stream paths are possible, for example more parallel to longitudinal axis 1112 (either proximal, or distal). In some embodiments an intersection region 1140 is external to the thrombectomy device 700 (not shown). The angle 1107 can be an angle in a range of about 30 degrees to about 75 degrees (e.g., about 45 degrees).

In some embodiments, the apertures of any of the thrombectomy devices described herein can include varied aperture shapes/configurations/sizes. For example, the sizes and/or shapes of the apertures can be varied to produce different fluid stream spray patterns, shapes, or cross-sections. For example, one or more of the apertures of a thrombectomy device may be sized, shaped, and configured to produce a solid fluid stream with a generally circular cross section. In other embodiments, one or more of the apertures may be sized, shaped, and configured to produce other fluid stream spray patterns including full cone spray patterns, spiral full cone spray patterns, hollow cone spray patterns, misting spray patterns, and/or flat fan spray patterns.

In some implementations, each of the apertures of a given thrombectomy device may have a different fluid stream spray pattern. For example, a thrombectomy device with four apertures may include a first aperture producing a first spray pattern (e.g., a solid fluid stream pattern), a second aperture producing a second spray pattern (e.g., a solid cone spray pattern), a third aperture producing a third spray pattern (e.g., a hollow cone spray pattern), and a fourth aperture producing a fourth spray pattern (e.g., a flat fan spray pattern). The number of different spray patterns implemented in a thrombectomy device is limited only by the number of apertures in the device and the number of different spray patterns available.

In other implementations, two or more apertures of a given thrombectomy device may produce the same fluid stream spray pattern, and other apertures may produce different spray patterns. For example, a thrombectomy device with four apertures may include first and second apertures producing a first fluid stream spray pattern (e.g., a solid fluid spray pattern), a third aperture producing a second fluid stream spray pattern (e.g., a hollow cone spray pattern), and a fourth aperture producing a third fluid stream spray pattern (e.g., a flat fan spray pattern). Alternatively, pairs of apertures may produce the same spray pattern (e.g., first and second apertures producing a first spray pattern and second and third apertures producing a second spray pattern).

In some embodiments, first opposing or intersecting apertures may be configured to produce one spray pattern while second opposing or intersecting apertures may be configured to produce another spray pattern. For example, referring to FIG. 8A, one embodiment shows three apertures with a single intersection point 840.

In other embodiments, first opposing or intersecting aperture spray patterns may be configured to produce different spray patterns. For example, one embodiment may include four apertures with a single intersection point. In this example, a first pair of opposing apertures may produce different spray patterns (e.g., a solid stream spray pattern and a hollow cone spray pattern) and a second pair of apertures may also produce different spray patterns. Alternatively, a thrombectomy device has four apertures with two intersection points. In this example the pair of apertures intersecting at the first intersection point may produce different spray patterns, and the other pair of apertures intersecting at the second intersection point may produce different spray patterns. In some embodiments, the volume of fluid associated with the intersections of spray patterns changes as a function of the intersection of the same or different spray patterns.

The size of the apertures, and thus the volume of fluid delivered and/or the cross-section of the fluid steams, may also be varied within a thrombectomy device. For example, a thrombectomy device may have any number of apertures with any number of aperture sizes. In some examples, the total amount of apertures is divided into groups of two, three, four, or five groups of apertures, each group having a different aperture size. In one example, opposing or intersecting pairs of apertures may have the same size aperture, or may have different sized apertures. Any combination of aperture sizes and groupings is contemplated. The size of the aperture may affect the shape or size of the fluid stream spray pattern. For example, a smaller sized aperture may result in a smaller spread on the spray pattern, while a larger sized aperture may result in a wider spread on the spray pattern.

Various irrigation and aspiration schemes are contemplated. In one embodiment, the fluid streams and or the aspiration are pulsed between on and off. The pulsing can be automatically controlled, or manually controlled. The timing of the pulses can be consistent or irregular. In some embodiments, aspiration remains on while the irrigation is pulsed. In other embodiments, the aspiration is pulsed while the irrigation remains on. The aspiration and irrigation can be pulsed in relation to another. For example, aspiration can be pulsed on when the irrigation is pulsed on. Or the aspiration can be turned off when the irrigation is pulsed on (or vis versa).

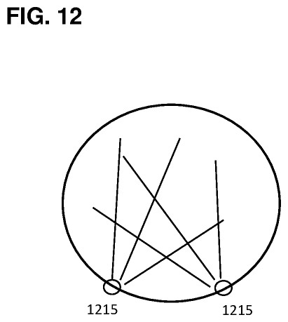

In one embodiment, referring to FIG. 12, apertures 1215 can be specially shaped to enable directional fluid streams, such as off-axis, tangential, etc. FIG. 12 shows off-axis or tangential directional fluid streams according to one embodiment. In some examples, the apertures can be controlled, adjusted, or configured to be able to produce streams in different directions. For example, the size or shape of the aperture can be changed to result in a changed fluid stream direction or size.

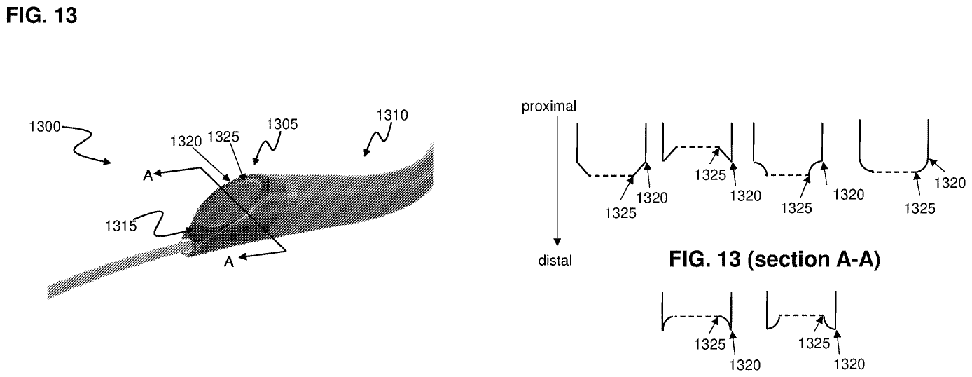

In some embodiments, an outer edge of the distal end can be shaped or formed to produce a desired effect, such as an atraumatic interaction with a vessel wall. FIG. 13 illustrates an example of a thrombectomy device 1300 having a distal portion 1305 and a proximal portion 1310. The device can further include a distal outer edge 915 having an outer diameter (OD) 1320 and inner diameter (ID) 1320. The distal outer edge 1315 of the thrombectomy device can have numerous shapes or designs as indicated by cross-section A-A, including:

Beveled distal outer edge 1315 with OD 1320 proximal to ID 1325.

Beveled distal outer edge 1315 with OD 1320 distal to ID 1325.

Concave distal outer edge 1315 with OD 1320 proximal to ID 1325.

Concave distal outer edge 1315 with OD 1320 distal to ID 1325.

Convex distal outer edge 1315 with OD 1320 proximal to ID 1325.

Convex distal outer edge 1315 with OD 1320 distal to ID 1325.

In some embodiments, the distal outer edge can include two or more planes or bevels or curvatures (not shown). In some examples, the distal outer edge is shaped and configured to skip or skim along the lumen wall including along any adhered or calcified material without digging in, gouging, or otherwise catching on the lumen wall or the adhered material. In other embodiments, the distal outer edge is shaped and configured to scrape or remove any adhered or calcified material.

The guidewires and guidewire lumens of the present disclosure can be configured in various ways. In one example, the device can be configured as a rapid exchange device in which the guidewire lumen maintains the annular space for fluid delivery, maintains pushability, and allows for torquing at large angles (e.g., more than 45 degrees).

In some embodiments, the guidewire can be fixed at the distal end, but free at the proximal end.

In other embodiments, the guidewire can be entirely fixed.

In some embodiments a thrombectomy device according to the present disclosure can be selectively stiffened and/or steered.

Selectively stiffen by:
i. generating a fluid column by irrigation alone. For example a “leaky” catheter. Optionally aspirate to compensate for introduced irrigant;
ii. blocking off aspiration lumen (in particular at the irrigation aperture) to selectively stiffen;
with obturator and/or (additional) inner catheter moved axially to selectively block/expose apertures.

Steering:
i. using blocker (as above) to vacuum down upon;
ii. inflating (additionally sheath includes spines on one aspect (side));
inflate, spines expand (large curve); deflate, spines contract (tighten curvature).
iii. using interference between one or more features on the guidewire, and features integral to the guidewire lumen (for examples see PCT/US2022/075986, filed Sep. 5, 2022, incorporated by reference herein).

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate shaft;
at least one aspiration lumen in the elongate shaft;
a funnel disposed at or near a distal end of the elongate shaft; and
an expandable scaffold assembly deployable distally from the funnel, the scaffold assembly being configured to capture clots;
wherein an axial position of the scaffold assembly relative to the funnel is adjustable to bring the scaffold assembly near or into contact with the funnel to aspirate clots in the scaffold assembly through the funnel.

2. The system of claim 1, further comprising one or more tethers coupled to the scaffold assembly.

3. The system of claim 1, wherein the one or more tethers are routed internally to the elongate shaft.

4. The system of claim 1, wherein the one or more tethers are routed internally to the funnel.

5. The system of claim 1, wherein the one or more tethers are routed externally to the elongate shaft.

6. The system of claim 1, wherein the one or more tethers are routed externally to the funnel.

7. The system of claim 2, wherein the one or more tethers are configured to adjust the axial position of the scaffold assembly relative to the funnel.

8. The system of claim 1, wherein the funnel is configured to engage with the scaffold assembly.

9. The system of claim 8, wherein the funnel is configured to be inserted into the scaffold assembly.

10. The system of claim 8, wherein the scaffold assembly is configured to be inserted into the funnel.

11. The system of claim 8, wherein a proximal opening of the scaffold assembly is configured to mate with the funnel.

12. The system of claim 1, wherein the thrombus removal device is configured to produce one or more fluid streams within the funnel to interact with clots in the funnel or scaffold assembly.

13. The system of claim 1, wherein the scaffold assembly is configured to evert into the funnel.

14. The system of claim 1, wherein manipulation of one or more tethers coupled to the scaffold is configured to evert the scaffold assembly into the funnel.

15. The system of claim 1, wherein the scaffold assembly comprises a superelastic material.

16. The system of claim 1, wherein the scaffold assembly is configured to self-expand from a delivery configuration to a deployed configuration.

17. A thrombectomy method, comprising:
positioning a thrombectomy device near a target thrombus in a patient;
deploying a scaffold assembly from the thrombectomy device;
capturing the target thrombus in the scaffold assembly;
positioning a funnel of the thrombectomy device near or in contact with the scaffold assembly; and
aspirating the target thrombus from the scaffold assembly through the funnel with the thrombectomy device.

18. The method of claim 17, wherein the thrombectomy device is positioned proximal to the target thrombus.

19. The method of claim 17, wherein the thrombectomy device is positioned distal to the target thrombus.

20. The method of claim 17, wherein the scaffold assembly is deployed distal to the target thrombus.

21-51. (canceled)
